(19) United States
(12) Patent Application Publication
(10) Pub. No.: US 2023/0000955 A1
(43) Pub. Date: Jan. 5, 2023

(54) THROMBOMODULIN FUNCTIONAL DOMAINS FOR USE IN PROMOTING OSTEOBLAST FUNCTIONS AND BONE HEALING

(71) Applicant: BLUE BLOOD BIOTECH CORP., Taipei City (TW)

(72) Inventors: Hua-Lin WU, Taipei City (TW); Tsung-Lin CHENG, Kaohsiung City (TW); Guey-Yurh SHI, Taipei City (TW)

(21) Appl. No.: 17/780,976

(22) PCT Filed: Dec. 15, 2020
(86) PCT No.: PCT/US20/65064

§ 371 Date: May 28, 2022

Related U.S. Application Data
(60) Provisional application No. 62/951,490, filed on Dec. 20, 2019.

(51) Int. Cl. A61K 38/36; A61P 19/08

(52) U.S. Cl. CPC A61K 38/366; A61P 19/08

(57) ABSTRACT

Thrombomodulin functional domains for use in promoting osteoblast functions and bone healing. Methods for stimulating bone regeneration, bone augmentation, treating a bone loss, a bone disorder and/or treating a bone-related disorder are disclosed. The method comprises administering, to a subject in need thereof a therapeutically effective amount of (I); a biologically, active recombinant polypeptide comprising, an amino acid sequence that is at least 80% identical to domains 2 and 3 of human thrombomodulin (TMD23); (II): an isolated polypeptide comprising recombinant TMD23 (rTMD23) or a biologically active recombinant variant thereof; or recombinant TMD23 (rTMD23) or a biologically active recombinant variant thereof, wherein the polypeptide is free of or lacking amino acid residues from domains 4 and 5 of the human thrombomodulin, and further wherein the biologically active variant thereof comprises an amino acid sequence that is at least 80% identical to the TMD23.

### FIELD OF THE INVENTION

The present invention relates generally to treating bone loss, promoting bone formation and bone fracture repair (osteoinduction, osteoaugmentation), and more specifically to methods for stimulating osteoblastogenesis and inhibiting osteoclastogenesis.

### BACKGROUND OF THE INVENTION

Bone remodeling process involves resorption of the mineralized bone by osteoclasts, followed by formation of bone matrix and mineralization by osteoblasts. Under stimulation of growth factors and vitamin D3, mesenchymal stem cells are converted into osteoprogenitor cells and osteoblasts to initiate osteogenesis. The osteoblasts secret type I collagen and calcium deposit to form osteoid, which is further mineralized to form bone tissues. On the other hand, under stimulation of macrophage colony-stimulating factor 1 (M-CSF) and receptor activator of nuclear factor kappa B (NFκB) ligand (RANKL), macrophages are transformed to form osteoclasts, which are responsible for bone resorption process called osteoclastogenesis. The balance between the bone resorption and formation processes is essential for maintaining homeostasis of the bone.

Osteoporosis is a disease that occurs when the mass or density of the bone is decreased because of an imbalance between the remodeling activities of osteoclasts and osteoblasts. Medications available for the prevention and/or treatment of osteoporosis include bisphosphonatcs, calcitonin, estrogen (hormone therapy), estrogen agonists, parathyroid hormone (PTH) analog, RANK ligand (RANKL) inhibitor, and tissue-selective estrogen complex (TSEC). These drugs have side effects and cannot stimulate osteoblastogenesis and inhibit osteoclastogenesis simultaneously.

Human thrombomodulin (TM) is a transmembrane protein and its structure consists of 5 domains, including a NH2-terminal lectin-like domain (D1), a domain with 6 epidermal growth factor (EGF)—like structures (D2), an O-glycosylation site—rich domain (D3), a transmembrane domain (D4), and a cytoplasmic tail domain (D5). Cheng et al. reported that TMD1 can inhibit osteoclastogenesis by reducing proinflammatory high-mobility group box 1 (HMGB1) signaling (“Myeloid thrombomodulin lectin-like domain inhibits osteoclastogenesis and inflammatory bone loss” Sci Rep. 2016; 6:28340).

U.S. Pat. No. 9,968,660 discloses methods of bone regeneration or bone augmentation.

### SUMMARY OF THE INVENTION

In one aspect, the invention relates to use of a biologically active recombinant polypeptide comprising an amino acid sequence that is at least 80% identical to domains 2 and 3 of human thrombomodulin (TMD23) in the manufacture of a medicament for stimulating bone regeneration, bone augmentation, treating a bone loss, a bone disorder and/or a bone-related disorder in a subject in need thereof, wherein the polypeptide is free of or lacking amino acid residues from domains 4 and 5 of the human thrombomodulin.

In another aspect, the invention relates to use of an isolated polypeptide comprising recombinant domains 2 and 3 of human thrombomodulin (rTMD23) or a biologically active recombinant variant thereof in the manufacture of a medicament for stimulating bone regeneration, bone augmentation, treating a bone loss, a bone disorder and/or a bone-related disorder in a subject in need thereof, wherein the biologically active variant thereof comprises an amino acid sequence that is at least 80% identical to the TMD23, and further wherein the polypeptide is free of or lacking amino acid residues from domains 4 and 5 of the human thrombomodulin.

In one embodiment, the polypeptide comprises amino acid residues of TMD23.

In another embodiment, the polypeptide comprises amino acid residues from 224Ala to 497Ser, or from 1Ala to 497Ser, of SEQ ID NO:1.

In another embodiment, the polypeptide comprises the amino acid sequence of SEQ ID NO: 3.

In another embodiment, the polypeptide is a biologically active recombinant TMD23 variant and comprises the amino acid sequence of SEQ ID NO: 3.

In another embodiment, the polypeptide further comprises amino acid residues of domain 1 from the human thrombomodulin.

In one embodiment, the polypeptide consists essentially of domains 2 and 3, or domains 1, 2 and 3, of human thrombomodulin.

In another embodiment, the recombinant TMD23 variant consists of the amino acid sequence of SEQ ID NO: 3.

In another embodiment, the polypeptide comprises an amino acid sequence selected from the group consisting of SEQ ID NO: 2, SEQ ID NO: 3, SEQ ID NO: 4, and SEQ ID NO: 5.

In another embodiment, the polypeptide or the biological active variant comprises amino acid residues that is at least 85%, or at least 90%, or at least 95% identical to rTMD23.

Further in another aspect, the invention relates to use of recombinant domains 2 and 3 of human thrombomodulin (rTMD23) or a biologically active recombinant variant thereof in the manufacture of a medicament for stimulating bone regeneration, bone augmentation, treating a bone loss, a bone disorder and/or a bone-related disorder in a subject in need thereof, wherein the biologically active variant thereof comprises an amino acid sequence that is at least 80% identical to the human TMD23.

In another embodiment, the recombinant variant of rTMD23 comprises the amino acid sequence of SEQ ID NO: 3.

In another embodiment, the recombinant TMD23 variant consists of the amino acid sequence of SEQ ID NO: 3.

In another embodiment, the bone disorder is a disease or condition selected from the group consisting of osteoporosis, bone loss, failure to achieve optimal bone formation, failure to achieve optimal bone fracture healing, low peak bone mass attainment during skeletal growth, impaired new bone formation, and any combination thereof.

In another embodiment, the bone-related disorder is at least one selected from the group consisting of hyperparathyroidism, hyperparathyroidism-related bone mass reduction, hyperparathyroidism-related decrease in bone mass density, osteopenia, bone loss, inflammatory bone loss, osteoporosis, old-age osteoporosis, post-menopausal osteoporosis, glucocorticoid-induced osteoporosis, osteonecrosis of the jaw, Paget's disease, and hyperphosphatemia.

Further in another embodiment, the use as aforementioned is in combination with use of bone grafting materials in the manufacture of a medicament for stimulating new bone formation. Bone graft materials provides structural stability and linkage and stimulate osteogenesis and bone healing in fractures.

The polypeptide or the biologically active recombinant variant of rTMD23 as aforementioned may be used in the manufacture of a medicament for treating a bone-related disorder, stimulating bone repair, promoting engraftment of a bone implant, or stimulating osteoblast proliferation.

In one embodiment, the medicament is in a dosage form suitable for application to a body site in need of a bone augmentation.

The body site in need of the bone augmentation is one selected from the group consisting of sinus lift, bone graft, and ridge expansion.

The medicament may be in gel, cream, paste, lotion, spray, suspension, solution, dispersion salve, hydrogel or ointment forms.

In another embodiment, a body site in need of bone augmentation is selected from a site that needs bone repair, bone fracture healing, or spinal fusion.

In one embodiment, a body site in need of the bone augmentation is dental implant placement.

The medicament may be in a dosage form containing a human daily dose of the polypeptide as aforementioned in a unit dose ranging from 100 ng/kg to 100 mg/kg×(0.025 in kg/human weight in kg)^{0.33}.

The invention also relates to methods for stimulating bone regeneration, bone augmentation, treating a bone loss, a bone disorder and/or a bone-related disorder. The method comprises administering to a subject in need thereof a therapeutically effective amount of (I): a biologically active recombinant polypeptide comprising an amino acid sequence that is at least 80% identical to TMD23; (II): an isolated polypeptide comprising a recombinant TMD23 (rTMD23) or a biologically active recombinant variant thereof; or (III): a recombinant TMD23 (rTMD23) or a biologically active recombinant variant thereof, wherein the polypeptide is free of or lacking amino acid residues from domains 4 and 5 of human thrombomodulin, and further wherein the biologically active variant thereof comprises an amino acid sequence that is at least 80% identical to the TMD23.

Alternatively, the invention relates to (I): a biologically active recombinant polypeptide comprising an amino acid sequence that is at least 80% identical to human TMD23; (II): an isolated polypeptide comprising a recombinant TMD23 (rTMD23) or a biologically active recombinant variant thereof; or (III): a recombinant TMD23 (rTMD23) or a biologically active recombinant variant thereof, for use in stimulating bone regeneration, bone augmentation, treating a bone loss, a bone disorder and/or a bone-related disorder in a subject in need thereof, wherein the polypeptide is free of or lacking amino acid residues from domains 4 and 5 of the human thrombomodulin, and further wherein the biologically active variant thereof comprises an amino acid sequence that is at least 80% identical to the TMD23.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

FIGS. 1A-B show recombinant TMD23 (rTMD23) and TMD123 promote cell migration in MG63 cells. MG63 cells were loaded into the up-compartment and various concentrations of rTMD23 or TMD123 as indicated were loaded in the low-compartment. After 4 hours of incubation, membrane was stained with Liu stain. Migrated cells were counted after taking photography. **P<0.01.

FIG. 2A shows recombinant TMD23 (rTMD23) enhanced cell proliferation in MG-63 cells. **P<0.01; ***P<0.001.

FIG. 2B shows recombinant TMD23NA, a variant of rTMD23, enhanced cell proliferation in MC3T3-E1 cells. *P<0.05 compared to the absorbance at 0 ng/ml of rTMD23NA.

FIG. 3 shows recombinant TMD23 (rTMD23) enhanced cell mineralization in MG-63 cells. Cells were incubated with various concentration of rTMD23 (0, 10, 25, 50 ng/mL) for 28 days. The cell were fixed by 10% formaldehyde for 20 min and then washed with PBS twice right before staining. Alizarin Red S (40%) was added at 1000 μL/well in each well for 20 min, follow by ddH₂O rinse at least five times. The photo was taken by 10 times magnitude inverted fluorescence microscope, and relative intensity was measured by ImageJ software. ***P<0.001.

FIGS. 4A-B show inhibition of RANKL-induced osteoclast formation by rTMD123 on RAW264.7 cells. (4A) The RAW264.7 cells were cultured for 4 days in the presence of RANKL (30 ng/ml) and M-CSF (20 ng/ml) with the indicated concentrations of rTMD123. Multinucleated osteoclasts were visualized using TRAP staining. (4B) TRAP⁺ OCs were counted. ***P<0.001 (versus the MCSF/RANKL-treated group). Data are expressed as mean±SD and are representative of at least three experiments.

FIGS. 5A-D show inhibition of RANKL-induced osteoclast formation in RAW264.7 cells by rTMD23 (5A-B) and the variant thereof (5C-D), respectively. The RAW264.7 cells were cultured for 4 days in the presence of RANKL and M-CSF with the indicated concentrations of rTMD23 or the variant rTMD23NA. Multinucleated osteoclasts were visualized using TRAP staining. The number of TRAP′ OCs in each well were counted. Data are expressed as mean±SD and are representative of at least three experiments.***P<0.001 (versus the MCSF/RANKL-treated group).

FIGS. 6A-B show the effect of rTMD123 on resorption pit formation. (6A) RAW264.7 cells were cultured on Corning Osteoassay 96-well plates with different concentrations of rTMD123 in the presence of MCSF (20 ng/ml) and RANKL (30 ng/ml) for 4 days. After incubation, resorbed lacunae on the plate were visualized using light microscopy. (6B) The percentages of resorbed area relative to the control group were calculated using Image J software. ***P<0.001 (compared with MCSF/RANKL-treated group).

FIGS. 7A-B show recombinant TMD23 (rTMD23) enhanced calvaria healing in STZ mice. Mice were injected with 50 mg/kg streptozotocin (STZ) per day for at least five days. Blood glucose was measured 3 days after the last dose of injection. Then the calvaria surgery was conducted to create a 4 mm-diameter hole. (7A) rTMD23 (0, 10, 100 μg/kg) was locally subcutaneously injected once on day 7 (week 1) after surgery and the micro CT (μCT) images were taken at week 0 and week 8. (7B) For data quantification, the term “TV” stands for the total volume of the hole and the term “By” stands for the regrowth bone volume at week 8. **P<0.01.

FIGS. 8A-F show the effect of the TMD1 and TMD23 on calvarial bone healing. (8A) Cell migration, (8B) proliferation and (8C) mineralization of MG63 cells treated with the indicated dose of rTMD1. (8D) Calvarial bone healing of TM wild-type TM^{wt/wt} and TM domain 1 (TMD1)-deleted TM^{LeD/LeD} mice. (8E) μCT scanning and (8F) HE stain were used to evaluate the effect of vehicle, dichloroisocoumarin (DCI) and DCI plus rTMD23 or rTMD1 (100 μg/kg) on bone healing after a calvarial defect was created in mice. N=5 per group. Black bar=100 μm. White bar=1 mm. Dotted line indicates the section region. ***P<0.001.

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. Referring to the drawings, like numbers indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention. Additionally, some terms used in this specification are more specifically defined below.

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The term “rTMD23” refers to recombinant domains 2 and 3 of thrombomodulin. Thrombomodulin (TM) is a cell membrane-bound glycoprotein composed of five domains, including a N-terminal lectin-like domain (D1), 6 epidermal growth factor (EGF) repeats (D2), a serine-threonine-rich region (D3), a transmembrane domain (D4) and a short cytoplasmic tail (D5). A mature human TM without signal peptide has 557 amino acid residues and comprises the amino acid sequence of SEQ ID NO: 1.

By at least 80% it meant all integer unit amounts within the range of 80% to 100% are specifically disclosed as part of the invention. Thus, 80%, 81%, 82%, 83%, 84, 85% . . . 88%, 89%, 90%, 91%, 92%, 93%, 94%, 95%, 96%, 97%, 98%, 99% and 100% unit amounts are included as embodiments of this invention.

The term “an amino acid sequence that is at least 80% identical to TMD23” shall encompass wild-type rTMD23 and bioactive recombinant variants thereof.

The terms “bioactive” and “biologically active” are interchangeable.

As used herein, the term “a biologically active recombinant variant thereof” shall generally mean a biologically active recombinant variant form of thrombomodulin domains 2 and 3 (TMD23).

By “biologically active recombinant variant of rTMD23” it meant a variant that possesses bioactivity as wild-type rTMD23, wherein the bioactivity is at least one selected from the group consisting of promoting cell proliferation and migration in osteoblasts, enhancing cell mineralization in osteoblasts, inhibiting osteoclastogenesis, inhibiting bone resorption activity, promoting bone regeneration, promoting osteoblastic functions and bone healing, and any combination thereof.

The terms “a biologically active variant form of recombinant TMD23”, “a biologically active recombinant human TMD23 variant”, “a bioactive recombinant human TMD23 variant polypeptide” and “human rTMD23 variant” are interchangeable.

As used herewith, the terms “a thrombomodulin variant”, “a variant of thrombomodulin” and “a mutant thrombomodulin” are interchangeable.

A variant of thrombomodulin shall generally mean a mutant thrombomodulin with amino acid residues variations from wild type thrombomodulin domains but retaining biological activities of the wild type thrombomodulin domains in promoting wound healing effect. Such thrombomodulin variants, rTMD23NA and rTMD123NA, have been disclosed in U.S. Pat. No. 9,156,904 B2, all of which are incorporated herein by reference with their entireties.

The term “rTMD23NA” refers to a recombinant TMD23 variant with two substitutions Asn364Ala and Asn391Ala. The rTMD23NA is a mutant with substitutions of Asn by Ala at residues 364 and 391 of wild-type mature human thrombomodulin (SEQ ID NO: 1).

The TMD123NA and rTMD23NA each are variants with two putative N-glycosylation Asn residues, N364 and N391 of TMD23, being mutated to Ala by a site-directed mutagenesis technique. The recombinant mutant rTMD23NA has little or no protein C activation activity. Preferably, a variant of thrombomodulin comprises and/or possesses the following features: (i) an amino acid sequence that is at least 80% identical to SEQ ID NO: 1 (557 aa); (ii) two amino acid residue substitutions Asn364Ala and Asn391A1a; and (iii) little or no protein C activation activity.

The term “treating”, or “treatment” refers to administration of an effective amount of a therapeutic agent to a subject, who has a disease, or a symptom or predisposition toward such a disease, with the purpose to cure, alleviate, relieve, remedy, ameliorate, or prevent the disease, the symptoms of it, or the predispositions towards it.

The terms “bone grafting materials” and “bone graft materials” are interchangeable. Bone grafting materials are known to provide structural stability and linkage and stimulating osteogenesis and bone healing in fractures.

The term “associated with” shall generally mean “related to” or “being related to”. Thus, “hyperparathyroidism associated with bone mass reduction” shall mean “hyperparathyroidism related to bone mass reduction”.

The “Guidance for Industry and Reviewers Estimating the Safe Starting Dose in Clinical Trials for Therapeutics in Adult Healthy Volunteers” published by the U.S. Department of Health and Human Services Food and Drug Administration discloses “a human equivalent dose” may be obtained by calculations from the following formula:

HED=animal dose in mg/kg×(animal weight in kg/human weight in kg)^{0.33}.

Sequence listing: SEQ ID NO: 1, wild-type full-length TM amino acid sequence without signal peptide (557 aa); domain 1, 1Ala-223Gly; domain 2, 224Ala-462Cys; domain 3, 463Asp-497Ser; domain 4, 498Gly-521Leu; domain 5, 522Arg-557Leu; SEQ ID NO: 2, rTMD23 sequence (274aa); SEQ ID NO: 3, rTMD23NA sequence (274aa); SEQ ID NO: 4, rTMD123 sequence (497aa); SEQ ID NO: 5, TMD123NA sequence (497aa).

Abbreviation: TM, thrombomodulin; rTM, recombinant thrombomodulin; rTMD23, recombinant thrombomodulin domains 2 and 3; TMD4, thrombomodulin domain 4; TMD5, thrombomodulin domain 5; Asn (N), Asparagine; Ala (A), Alanine.

The invention relates to the discovery of truncated thrombomodulin (TM) proteins comprising thrombomodulin domains 2 and 3 (TMD23) or the variant thereof possessing bioactivities in inhibiting macrophages transformation to osteoclasts and promoting the formation of osteoblasts from mesenchymal stem cells. It was discovered that truncated TM proteins TMD23, TMD123 and the variant TMD23NA could increase osteoblastogenesis and inhibit osteoclastogenesis simultaneously. These bioactive TM domains can be applied to stimulate bone formation and inhibit bone loss.

The invention also relates to a recombinant protein TMD23, TMD123, or a bioactive variant thereof, for use in improving and treating osteoporosis. It was discovered that among recombinant TM fragments, TM domains 2 and 3 (TMD23) rather than the lectin-like domain (TMD1) promoted osteoblast functions and calvarial bone healing. The invention further relates to a truncated TM for use in stimulating osteoblast while inhibiting osteoclast either through systemic, local administration or in combination with bone graphing material.

### EXAMPLES

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

Materials and Methods

Expression and purification of recombinant TM domain and variant proteins. Preparation of rTMD23 and the variant rTMD23NA have been disclosed in U.S. Pat. No. 9,156,904 B2, which is incorporated herein by reference in its entirety. Briefly, the pCR3 or pPICZA vector (Invitrogen, Oregon) was used for the expression and secretion of recombinant TM domain proteins in the HEK293 or Pichia pastoris protein expression system. DNA fragments encoding TMD1, TMD23 and TMD123 were obtained as previously described (Shi et al. “Lectin-like domain of thrombomodulin binds to its specific ligand Lewis Y antigen and neutralizes lipopolysaccharide-induced inflammatory response” Blood. Nov. 1, 2008; 112(9):3661-70; Cheng et al. “Functions of rhomboid family protease RHBDL2 and thrombomodulin in wound healing” J Invest Dermatol. December 2011; 131(12):2486-94). Expressed recombinant proteins were applied to a nickel-chelating Sepharose column, and recombinant TM domain-containing fractions were eluted.

Cell culture and treatment. Osteoblast cell line MC3T3-E1, human osteoblast-like cell line MG63, and mouse macrophage cell line RAW264.7 (ATCC, Rockville, Md., USA) were used. All cells were cultured in Dulbecco's modified Eagle's medium (DMEM, Gibco, Md., USA) that contained 10% fetal bovine serum (FBS, Gibco).

Transwell cell migration assay. Cell migration was evaluated using a 24-well chemotaxis chamber with a membrane of 8-μm pore size. A cell suspension (5×10⁴ cells/100 μL of serum-free DMEM medium for MG63 cells) was added to the upper chamber and a recombinant TM domain(s) in a serum-free medium (600 μL) was added to the lower chamber. The chambers were incubated at 37° C. for 8 hours. Cells that were not migrated were wiped off with a cotton slab. The filter was developed using Liu's stain kit and the number of remaining cells were counted under a microscope.

Proliferation assay. MG63 cells were treated with various concentrations of rTMD23 for 1, 4, 7 days. The medium was replaced every 2-3 days. At the end, the amount of living cells in each well was measured with crystal violet assay. The cells in each well were washed by PBS twice right before staining. Crystal violet (0.5%, 300 μL) were added in each well for 15 min, followed by ddH₂O rinse at least five times. Acetic acid (10%, 300 μL) were added to dissolute the crystal violet. Then, taking 100 μL/well into a 96 well plate to get an OD595 measurement. A proliferation assay on MC3T3-E1 cells with various concentrations of a variant of TMD23 was performed. Briefly, MC3T3-E1 cells in 96-well plates were treated with rTMD23NA at various concentrations for 1, 3, 5 days. The number of living cells in each well was measured with WST-1 reagent. The cells in each well were washed by DMEM medium twice right before staining with 100 μL of 10% WST-1 in DMEM. The absorption at A_{450 nm}-A_{690 nm} of each well was measured.

Mineralization assay. MG63 cells were seeded in 6-well plates (5×10⁵ cells/well) and treated with recombinant TM domains for 28 days. The mineralized matrix was stained for calcium using Alizarin-red staining as previously described (Stanford et al. “Rapidly forming apatitic mineral in an osteoblastic cell line (UMR 106-01 BSP)” J Biol Chem. Apr. 21, 1995; 270 (16): 9420-8). Briefly, cells were washed with PBS, followed by fixation in ice-cold 70% ethanol for 1 hour. After washing, cells were stained with 40 mM Alizarin-red (pH 4.2) for 10 minutes at room temperature. Stained cells were further processed by five-times rinse with water and followed by a 15-minute wash in PBS with rotations to reduce nonspecific Alizarin-red stain. Images were photographed and relative intensity measured by ImageJ software.

Experimental animals and diabetic mice. Six to eight weeks old mice lacking TM lectin-like domain (TM^{LeD/LeD}) and wild-type C57BL/6 mice were used (Conway et al. “The lectin-like domain of thrombomodulin confers protection from neutrophil-mediated tissue damage by suppressing adhesion molecule expression via nuclear factor kappaB and mitogen-activated protein kinase pathways” J Exp Med. Sep. 2, 2002; 196(5):565-77). Diabetic mice were generated by a single intravenous injection of streptozocin (STZ) (150 mg/kg) dissolved in 50 mM citrate, pH 4.7. Control mice were injected with a citrate buffer. The glucose content was measured using a glucose kit (Randox, Crumlin, UK) according to the manufacturer's instructions. The blood glucose concentration of diabetic mice reached 413.9 mM (250 mg/dL).

Calvarial bone defect healing. Male mice were randomly assigned to designated groups. After anesthesia, a 4 mm-diameter defect on the calvaria was created and the wound was sutured. A combination of dichloroisocoumarin (DCI) (1 mM) or rTMD23 (0, 10, 100 μg/kg) and U0126 (20 μM) was subcutaneously injected once near the calvarial bone opening at week 1 after the surgery. Bone healing was monitored radiographically using μCT (Skyscan 1076, Antwerp, Belgium) and histologically at indicated time points (weeks 0 and 8) after the surgery. The μCT scanner was operated at 50 kV, 220 mA, 1.2 s exposure time, with a pixel size of 2.5 mm, 0.5 mm aluminum filter, yielding isotopic 17.09 mm voxel with 2.5 mm slice thickness through a rotation of 180″. To calculate the bone formation, a cylindrical volume of 4 mm diameter and 2 mm depth was chosen for the calvarial defect regeneration. The tomographic images were transformed into volumetric reconstruction using the NRecon program (Skyscan). The quantification of the removed and regenerated bone volume (mm³) performed by a 3D CT analyzer software (CTAN; Skyscan). For histological analysis, the specimens were decalcified in 10% EDTA for 1 week and then embedded in paraffin. Hematoxylin-Eosin (H&E) staining was performed using 10 mm-thick sections.

Statistical analysis. Statistical analysis was performed using Prism 7 (GraphPad software). Data are expressed as mean±SD. For comparisons of multiple groups, one-way analysis of variance (ANOVA) was used. For comparisons of multiple groups at different time points, two-way ANOVA was used. A value of P<0.05 was considered statistically significant.

Results

Both rTMD23 and rTMD123 Promote Cell Migration in Osteoblasts

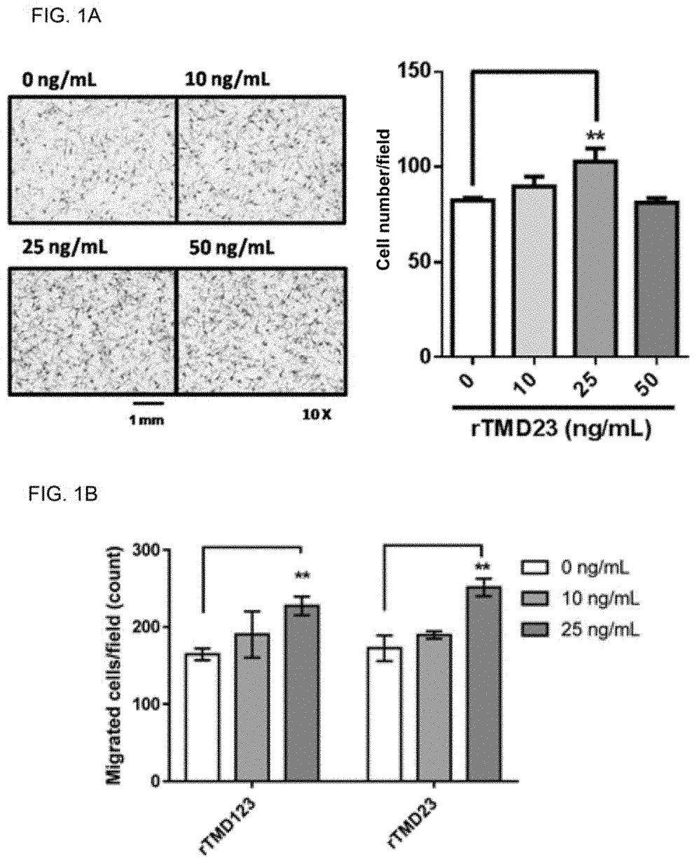

The cellular function of osteoblasts includes migration, proliferation and mineralization, Cells in serum free medium were added to the upper transwell chamber with 1% fetal bovine serum plus increasing doses of rTMD23 in the lower transwell chamber. After incubation for 4 hours at 37° C., the transwell membranes were removed and stained. The number of the cells that migrated through the membrane to the lower surface of the membrane was counted under a microscope. The cell migration assay revealed that both rTMD23 and rTMD123 increased cell migration of osteoblasts (FIGS. 1A-B).

Both rTMD23 and Bioactive Variants Thereof Promote Cell Proliferation in Osteoblasts

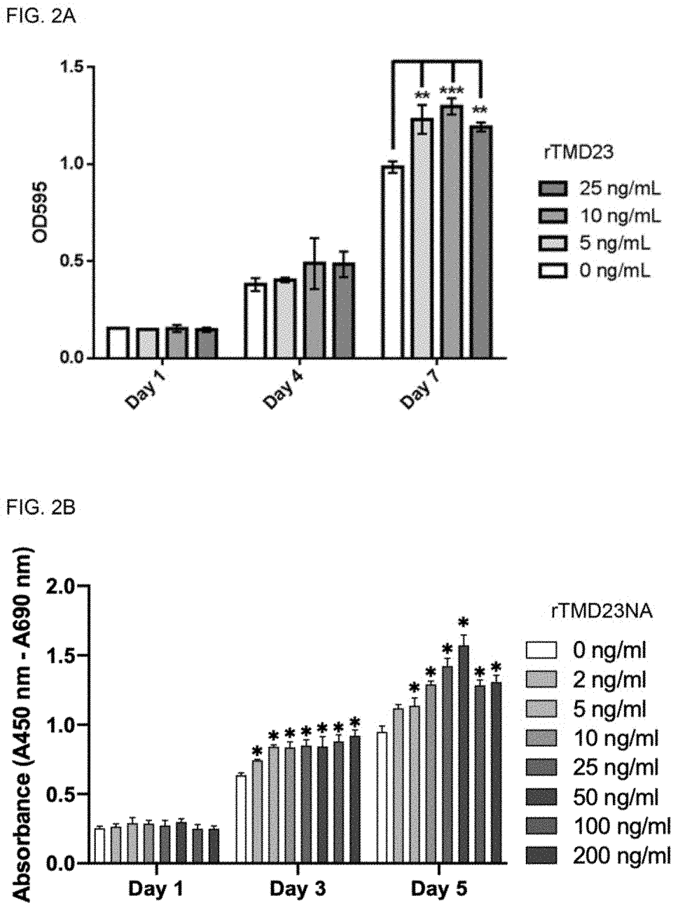

Human osteoblast-like cell line MG63 and osteoblast cell line MC3T3-E1 were used in the cell proliferation assay. Briefly, different concentrations of rTMD23 and rTMD23NA, which is a variant form of rTMD23, were respectively added to MG63 or MC3T3-E1 cell culture containing 1% FBS. The cell cultures were washed with PBS twice and stained with crystal violet. The OD595 of the cell lysate in 10% acetic acid was measured to obtain the relative cell count in each well. The results indicated that rTMD23 and the variant rTMD23NA could enhance cell proliferation in the human osteoblast-like cell line MG63 (FIG. 2A) and the osteoblast cell line MC3T3-E1 (FIG. 2B), respectively.

rTMD23 Enhances Cell Mineralization in Osteoblasts

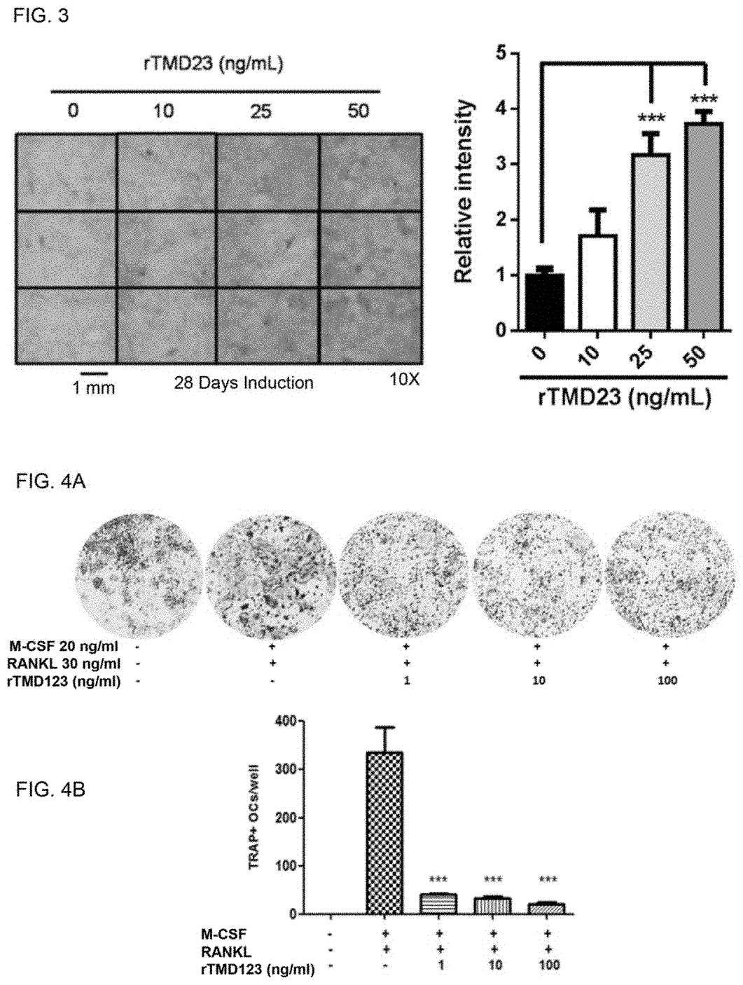

MG63 cells were incubated in a culture medium containing 1% FBS in the presence of rTMD23 at a concentration as indicated for 28 days. After being fixed with 10% formaldehyde, cells were stained with 40% alizarin red S. The results indicated that rTMD23 could promote mineralization in MG63 cells (FIG. 3).

rTMD123, rTMD23 and a Variant Thereof all Inhibit Osteoclastogenesis

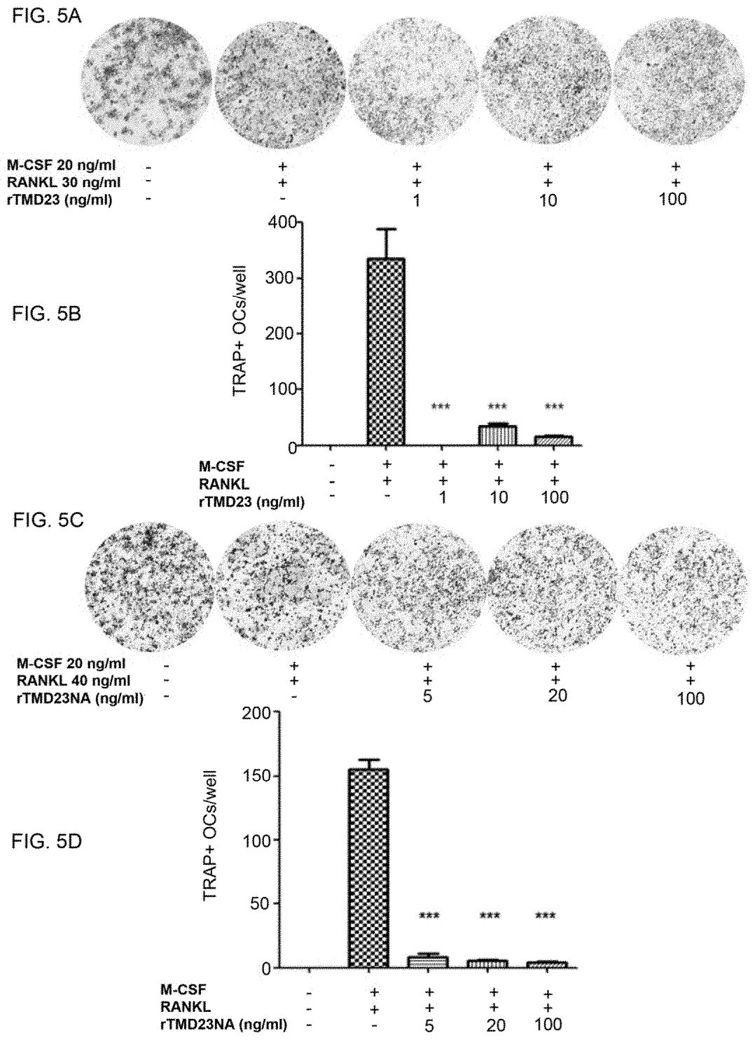

To induce osteoclastogenesis, RAW264.7 cells were cultured for 4 days in the presence of RANKL (30 ng/ml) and M-CSF (20 ng/ml) to induce the formation of TRAP positive osteoclasts. The multinucleated osteoclasts in each well were visualized using TRAP staining (FIG. 4A). The numbers of TRAP′ OCs formed in each well were counted (FIG. 4B). In the presence of rTMD123 the formation of TRAP positive osteoclasts was inhibited. Similar results were obtained with rTMD23 (FIG. 5A-B) and the variant rTMD23NA (FIGS. 5C-D). The results indicate that both rTMD23 and rTMD123 can inhibit osteoclastogenesis in vitro.

rTMD123 Inhibits Bone Resorption Activity in Osteoclasts

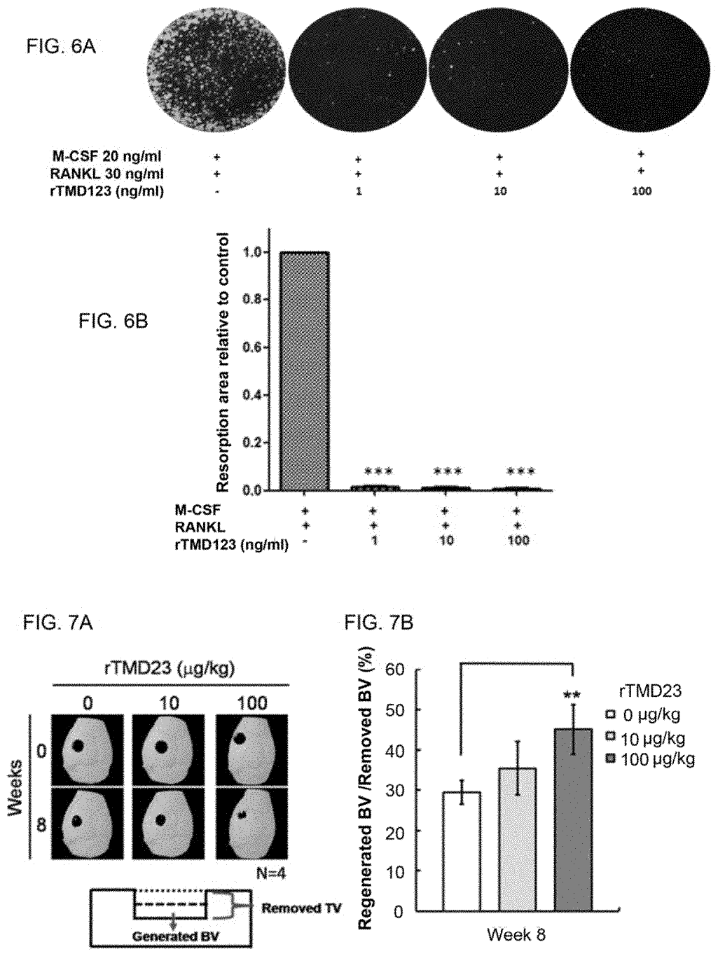

RAW264.7 cells cultured on Corning Osteoassay 96-well plates were induced to form osteoclasts with MCSF (20 ng/ml) and RANKL (30 ng/ml) in the presence of different concentrations of rTMD123. After incubation for 4 days, the resorbed lacunae on each well were visualized using light microscopy (FIG. 6A) and the resorbed area measured using Image J software. The percentages of the bone resorbed area were significantly decreased in the rTMD123-treated groups relative to the control group (FIG. 6B). The results indicate that rTMD123 can inhibit bone resorption activity in RAW264.7 cells.

rTMD23 Enhances Calvarial Bone Healing in Streptozotocin-Induced Diabetic Models

Mice were induced to have diabetics by injection with 50 mg/kg streptozotocin (STZ) for 5 days. A 4 mm-diameter hole was created in calvarial bone in each mouse. rTMD23 of various dosages was given once by subcutaneously injection near the calvarial bone opening at week 1 after the surgery. The micro CT images were taken at week 0 and week 8 (FIG. 7A). The bone volume that regenerated in 8 weeks was significantly increased in the rTMD23-treated group (FIG. 7B). The results indicate that rTMD23 can promote the regeneration of the bone. The mean bodyweight of the mice was 25 g.

rTMD23 but not rTMD1 Promotes Calvarial Bone Healing

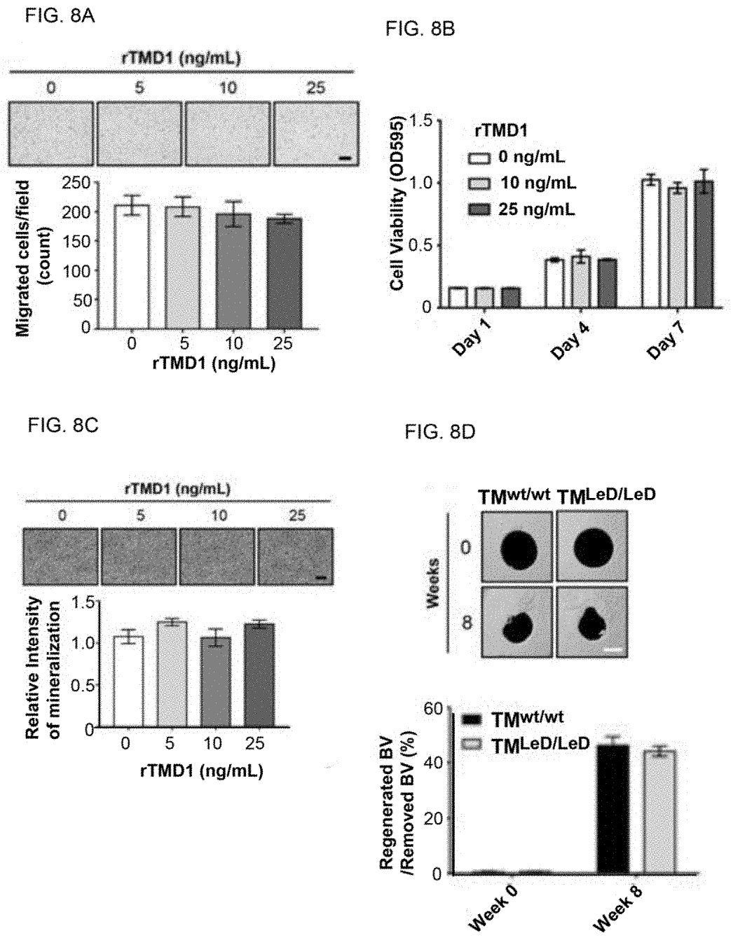

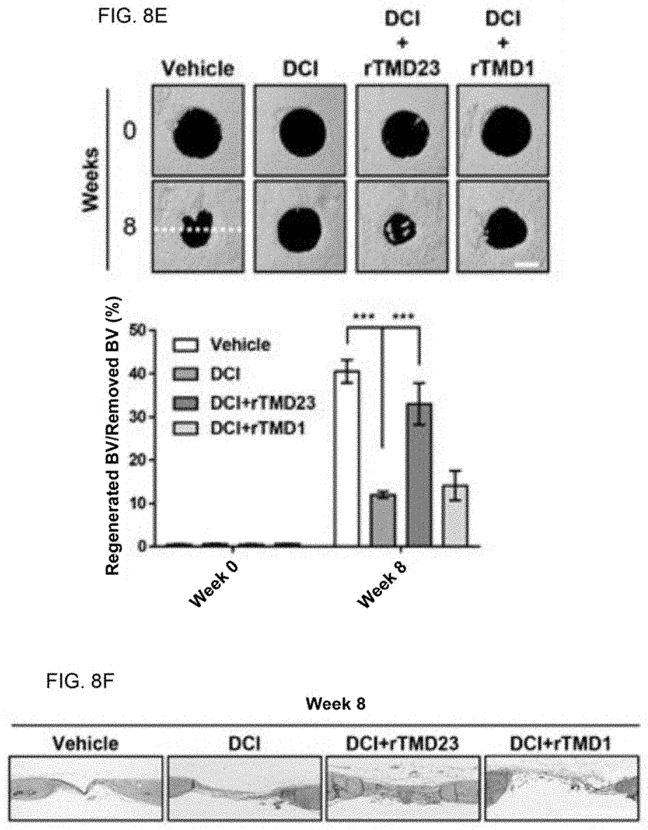

We further investigated whether TM lectin-like domain (TMD1) might also enhance cellular functions of osteoblasts. Functional assays showed that rTMD1 did not promote cell migration (FIG. 8A), or proliferation (FIG. 8B), or mineralization (FIG. 8C) in MG63 cells. Transgenic mice with deleted TMD1 (TM^{LeD/Led}) were used to further evaluate the effect of TMD1 on bone regeneration using the calvarial bone healing assay. As demonstrated by μCT, no significant difference of bone healing was found between the TM^{LeD/LeD} mice and the TM wild-type TM^{wt/wt} control mice at week 8 (FIG. 8D). These findings suggested that TMD1 cannot contribute to osteoblastic functions and bone healing. The calvarial bone healing assay was also used to evaluate the effect of TMD23 on bone healing. Both μCT measurements (FIG. 8E) and H&E staining (FIG. 8F) revealed that treatment with DCI to inhibit RHBDL2 and TM ectodomain shedding reduced regeneration of calvarial bone defect. However, this inhibitory effect could be rescued by rTMD23, but not by rTMD1. The results indicate that rTMD23 but not rTMD1 has the capacity to promote osteoblastic functions and bone healing.

In summary, rTMD23, rTMD123, or variants thereof, can promote migration, proliferation and mineralization in osteoblasts. As illustrated, TMD23 can promote bone formation by functioning as a chemoattractant to attract osteoblast and preosteoblast cells MG63 (a human osteoblast-like cell line). MG63 cells are phenotypically arrested at pre-osteoblast state and can be induced for mineralization. It was demonstrated that TMD23 can promote bone healing in STZ-induced diabetic mice. Macrophage can be induced to form multinuclear osteoclast in a process of osteoclastogenesis by stimulation with macrophage colony-stimulating factor 1 (MCSF-1) and RANK ligand (RANKL). TMD23, TMD123, or a bioactive variant thereof, can inhibit osteoclastogenesis of macrophages induced by MCSF-1 and RANK ligand (RANKL). As illustrated, rTMD23 can inhibit bone resorption by osteoclasts.

In conclusion, thrombomodulin domain 23-containing proteins, including TMD23, TMD123, and bioactive variants thereof, can inhibit osteoclastogenesis and promote osteoblastogenesis. TMD23, TMD123 or a bioactive variant thereof can be applied for prevention and treatment of bone loss diseases, conditions, or disorder, including osteoporosis, osteopenia, rheumatoid arthritis, arthritis and periodontitis, and for promoting bone regeneration, osteoaugmentation, treatment of bone injuries and promoting bone healing.

The foregoing description of the exemplary embodiments of the invention has been presented only for the purposes of illustration and description and is not intended to be exhaustive or to limit the invention to the precise forms disclosed. Many modifications and variations are possible in light of the above teaching.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

1. A method for stimulating bone regeneration, bone augmentation, treating a bone loss, a bone disorder and/or a bone-related disorder, comprising: administering to a subject in need thereof a therapeutically effective amount of a biologically active recombinant polypeptide comprising an amino acid sequence that is at least 80% identical to domains 2 and 3 of human thrombomodulin (TMD23), wherein the polypeptide is free of or lacking amino acid residues from domains 4 and 5 of the human thrombomodulin.

2. A method for stimulating bone regeneration, bone augmentation, treating a bone loss, a bone disorder and/or a bone-related disorder, comprising: administering to a subject in need thereof a therapeutically effective amount of an isolated polypeptide comprising recombinant domains 2 and 3 of human thrombomodulin (rTMD23) or a biologically active recombinant variant thereof, wherein the biologically active variant thereof comprises an amino acid sequence that is at least 80% identical to the domains 2 and 3 of the human thrombomodulin (TMD23), and further wherein the polypeptide is free of or lacking amino acid residues from domains 4 and 5 of the human thrombomodulin.

3. The method of claim 1, wherein the polypeptide comprises amino acid residues of domains 2 and 3 from the human thrombomodulin.

4. The method of claim 1, wherein the polypeptide comprises amino acid residues from 224Ala to 497Ser, or from 1Ala to 497Ser, of SEQ ID NO: 1.

5. The method of claim 1, wherein the polypeptide comprises the amino acid sequence of SEQ ID NO: 3.

6. The method of claim 1, wherein the polypeptide is a biologically active recombinant TMD23 variant and comprises the amino acid sequence of SEQ ID NO: 3.

7. The method of claim 1, wherein the polypeptide further comprises amino acid residues of domain 1 from the human thrombomodulin.

8. The method of claim 2, wherein the recombinant TMD23 variant consists of the amino acid sequence of SEQ ID NO: 3.

9. The method of any one of claim 1, wherein the polypeptide comprises an amino acid sequence selected from the group consisting of SEQ ID NO: 2, SEQ ID NO: 3, SEQ ID NO: 4, and SEQ ID NO: 5.

10. The method of claim 1, wherein the polypeptide or the biological active variant comprises an amino acid sequence that is at least 95% identical to rTMD23.

11. The method of claim 1, wherein the polypeptide or the biological active variant comprises amino acid residues that is at least 90% identical to rTMD23.

12. A method for stimulating bone regeneration, bone augmentation, treating a bone loss, a bone disorder and/or a bone-related disorder, comprising:
administering to a subject in need thereof a therapeutically effective amount of recombinant domains 2 and 3 of human thrombomodulin (rTMD23 or a biologically active recombinant variant thereof, wherein the biologically active variant thereof comprises an amino acid sequence that is at least 80% identical to the domains 2 and 3 of the human thrombomodulin.

13. The method of claim 12, wherein the recombinant variant of rTMD23 comprises the amino acid sequence of SEQ H) NO: 3.

14. The method of claim 1, wherein the bone disorder is a disease or a condition that is at least one selected from the group consisting of osteoporosis, bone loss, failure to achieve optimal bone formation, failure to achieve optimal bone fracture healing, low peak bone mass attainment during skeletal growth, and impaired new bone formation.

15. The method of claim 1, wherein the bone-related disorder is at least one selected from the group consisting of hyperparathyroidism, hyperparathyroidism-related bone mass reduction, hyperparathyroidism-related decrease in bone mass density, osteopenia, hone loss, inflammatory bone loss, osteoporosis, old-age osteoporosis, post-menopausal osteoporosis, glucocorticoid-induced osteoporosis, osteonecrosis of the jaw, Paget's disease, and hyperphosphatemia.

16. The method of claim 1, wherein the polypeptide is administered to the subject in need thereof in combination with administration of bone grafting materials to the subject in need thereof for stimulating new bone formation.

17. The method of claim 2, wherein the biological active variant comprises an amino acid residues that is at least 85% identical to rTMD23.

18. The method of claim 2, wherein the bone disorder is a disease or a condition that is at least one selected from the group consisting of osteoporosis, bone loss, failure to achieve optimal bone formation, failure to achieve optimal bone fracture healing, low peak bone mass attainment during skeletal growth, and impaired new bone formation.

19. The method of claim 12, wherein the bone disorder is a disease or a condition that is at least one selected from the group consisting of osteoporosis, bone loss, failure to achieve optimal bone formation, failure to achieve optimal bone fracture healing, low peak bone mass attainment during skeletal growth, and impaired new bone formation.

20. The method of claim 2, wherein the polypeptide is administered to the subject in need thereof in combination with administration of bone grafting materials to the subject in need thereof for stimulating new bone formation.


SEQUENCE LISTING (5 sequences)

SEQ ID NO: 1 - protein, 557 aa, Homo sapiens
APAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHSGLLIGISIASLCLVVALLALLCHLRKKQGAARAKMEYKCAAPSKEVVLQHVRTERTPQRL

SEQ ID NO: 2 - protein, 274 aa, Homo sapiens
AWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHS

SEQ ID NO: 3 - protein, 274 aa, Homo sapiens
AWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLAQTSYLCVCAEGFAPIPHEPHRCQMFCAQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHS

SEQ ID NO: 4 - protein, 497 aa, Homo sapiens
APAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHS

SEQ ID NO: 5 - protein, 497 aa, Homo sapiens
APAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLAQTSYLCVCAEGFAPIPHEPHRCQMFCAQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHS